(19) United States
(12) Patent Application Publication
(10) Pub. No.: US 2026/0029345 A1
(43) Pub. Date: Jan. 29, 2026

(54) POLYMER SEQUENCING APPARATUS

(71) Applicant: Armonica Technologies, Inc., Albuquerque, NM (US)

(72) Inventors: Steve Roy Julien Brueck, Albuquerque, NM (US); Xin Jin, Albuquerque, NM (US); Victor C. Esch, Albuquerque, NM (US)

(21) Appl. No.: 18/998,917

(22) PCT Filed: Jul. 28, 2023
(86) PCT No.: PCT/US2023/071183

§ 371 Date: Jan. 27, 2025

Related U.S. Application Data
(60) Provisional application No. 63/393,130, filed on Jul. 28, 2022.
(60) Provisional application No. 63/405,801, filed on Sep. 12, 2022.

(51) Int. Cl. G01N 21/64; G01N 21/65

(52) U.S. Cl. CPC G01N 21/6486; G01N 21/65

(57) ABSTRACT

The invention provides methods and device for determining the identity and order of units of large biopolymers. Device of the invention use electrical fields to translocate the molecule through the channel and pass the molecule, base-by-base, by an enhancement structure that enhances an incoming optical stimulus such as an electromagnetic field or wave. The presence of a DNA bases at the exit, adjacent the enhancement structure, has a characteristic effect in response to the enhanced optical stimulus, such as a characteristic fluorescence or Raman scattering. The characteristic effect of each base is read by the detector to determine the sequence of the molecule.

### TECHNICAL FIELD

This invention relates to reading the sequence of polymers.

### BACKGROUND

The information in DNA is stored as a code made up of four bases: adenine (A), guanine (G), cytosine (C), and thymine (T). A human genome includes about 3 billion bases of double-stranded DNA (dsDNA). The order, or sequence, of those bases along a strand determines the information available for building and maintaining an organism, similar to the way in which letters of the alphabet appear in order to convey meaning.

The total market for DNA sequencing was ˜$15.7B in 2020, growing at an 19.1% compound annual growth rate, and is expected to reach $38B by 2026. Assuming a 5% market share, this amounts to $1.9B in sales in 2026. Those estimates, based on extrapolations, illustrate an opportunity. The current approaches to next-generation sequencing (NGS) offer high throughput capability (e.g., Illumina, ABI SOLID, Ion Torrent, Complete Genomics/BGI, and Ultima Sequencing among others), but are limited to sequence reads of about 100 to a few hundred bases. Compared to the 3 billion bases of the human genome, those reads are extremely short. Short read platforms have major limitations, including that they are unable to: resolve haplotype-specific differences, provide a high quality de novo sequencing data for organisms, or unambiguously sequence highly repetitive regions and structural variants, which can amount to 5 to 15% of the human genome. Given those issues, it is not surprising that it was not until May of 2021 that a true telomere-to-telomere assembly of a complete human genome was announced. Nurk, 2021, The complete sequence of a human genome, BioRxiv 445798 and Nurk, 2022, The complete sequence of a human genome, Science 376 (6588): 44-53, both incorporated by reference.

### SUMMARY

The invention provides methods and device for determining the identity and order of units of large biopolymers. Device of the invention translocate a molecule such as a nucleic acid through a nanochannel and pass the molecule, one subunit after another, by an enhancement structure that enhances an incoming optical stimulus such as an electromagnetic field or wave. The identify of each subunit of the polymer (such as a base of a nucleic acid) as that submit passes by the enhancement structure exhibits a characteristic effect on the enhanced optical stimulus, such as a characteristic fluorescence or Raman scattering. The characteristic effect of each subunit may be read by a detector to determine the sequence of the molecule.

The invention provides methods and device for determining the identity and order of polymer units, particular for biological polymers such as nucleic acids. Methods and device of the invention make use of nanochannels to extend and linearize large biopolymers. Using DNA as an example, a long (e.g., tens of thousands of bases) single molecule of DNA can be pulled into a channel to essentially untangle and linearize the DNA molecule. Device of the invention use electrical fields to translocate the molecule through the channel and pass the molecule, base-by-base, by an enhancement structure that enhances an incoming optical stimulus such as an electromagnetic field or wave. In response to the optical stimulus, each base of the DNA emits a characteristic signal, which is read by a detector such a sensor or photodiode. Because the DNA molecule is pass by the enhancement structure in base-by-base order, and because each detected signal is characteristic of the identify of each base of the DNA molecule, the set of detected signals constitutes a read of the identity and order of the bases of the molecule. In fact, a connected computer system can write the detected signals to memory or a file as a sequence read or a sequence data file, which represents the sequence of bases of the DNA molecule.

The system may be used to read the identity and order of units of any suitable polymer. Preferred embodiments work with nucleic acids such as DNA. The optical stimulus can be from a pump illumination, laser, diode, or similar. The DNA molecule passes by (preferably emerges from an exit of the nanochannel) at a location adjacent an enhancement structure, which is preferably a metal-insulator-metal (MIM) stack that enhances the optical stimulus. The presence of a DNA bases at the exit, adjacent the enhancement structure, has an effect such as fluorescence, interference, or scattering in response to the enhanced optical stimulus. For example, in some embodiments, each base of DNA emits a characteristic fluorescence. In other embodiments, each base of DNA contributes to characteristic Raman scattering (e.g., surface-enhanced Raman scattering or similar). The characteristic effect of each base is read by the detector to determine the sequence of the molecule.

The present disclosure illustrates fabrication methods for such nanochannels and enhancement structures along with the platforms, covers, electrodes, and other features that may be included. Such devices of the invention are useful in methods of long-read, single-molecule sequencing techniques. In such methods, a solution of DNA may be loaded (e.g., pipetted) into an inlet or reservoir of the device. Electrodes drive a molecule of the DNA through a nanochannel and the enhancement structure is used to make a long (e.g., significantly more than tens of thousands of contiguous bases) sequence read from the DNA molecule. Such long-read sequencing may be used to quickly read genetic information that would be difficult to read by short-read, next generation sequencing (NGS) platforms. For example, methods and devices of the invention may be used to read entire chromosomes in a single instrument run, with haplotype phasing. Methods and devices of the invention are useful to discover, read, and map chromosomal rearrangements such deletions, insertions, translocations, inversions, and copy number variant or, more generally, structural variants. Thus methods and devices of the invention open up new applications for genetic sequencing in diverse fields such as diagnostics, agriculture, metagenomics, and biodiversity discovery.

Particular embodiments of the invention make use of a chip (optionally mounted on a separate, distinct carrier chip) through which at least one nanochannel ahs been fabricated. In one embodiment of this invention, a dsDNA strand is directed through a nanochannel, an enhancement structure is placed at the exit of the nanochannel to provide optical readout. A low frequency electric field is used to control the motion of the dsDNA molecule along the nanochannel and past the enhancement structure. This structure is suitable for data storage applications.

In a second embodiment of this invention, the nanochannel is packed with a small number of silica nanoparticles to slow the translocation of the dsDNA through the nanochannel.

In a third embodiment of this invention, a ssDNA strand is directed through a nanochannel, an enhancement structure is placed at the exit of the nanochannel to provide optical readout. A low frequency electric field is used to control the motion of the ssDNA molecule along the nanochannel and past the enhancement structure. This structure is suitable for DNA sequencing applications. In a fourth embodiment of this invention, the nanochannel is packed with a small number of silica nanoparticles to slow the translocation of the ssDNA through the nanochannel.

In a fifth embodiment of this invention, a section near the end of the nanochannel is filled with porous silica and the enhancement structure is placed atop this porous silica, forcing the ssDNA to pass by the hot spot at the edge of the enhancement structure.

In certain aspects, the invention provides a device for reading units of a polymer, the device comprising: a loading reservoir for receiving a sample that includes a polymer; a channel extending from the reservoir; electrodes operable to generate a field to drive the polymer from the reservoir and through the channel, wherein the polymer assumes an elongated conformation in the channel; an enhancement structure at an exit of the channel to enhance electromagnetic fields at the exit and excite fluorescence from the polymer. The device may include a detector to receive fluorescent or optical signals emitted from the polymer at the exit. further comprising a linear array of nanopillars at the exit of the channel and a second set of nanochannels and enhancement structures to provide a second readout of the data encoded in the polymer. Preferably the channel is one of a plurality of nanochannels. The polymer (e.g., DNA molecule) may be in the channel. The device may include a funnel structure and/or a nanopillar array at the reservoir for aiding in loading the DNA into the nanochannel. In some embodiments, the nanochannels are thirty to sixty nm in cross section dimensions and the length is five to ten mm.

In certain embodiments, the enhancement structure is elliptical in top down view and is positioned and rotated relative to the nanochannel axis to locate the hot spot of the enhancement structure close to the exit of the nanochannel. A section of the nanochannels may be loaded with silica nanoparticles to slow translocation of the DNA molecule. Preferably the silica nanoparticles are approximately uniform in size and chosen to fill the nanochannels with gaps between the particles and the nanochannel sidewalls of five nm or less. Optionally a section of the nanochannels is filled with a porous silica matrix to slow the DNA translocation.

A computing system may be operably coupled to the detector and operable to read the signals and write and store the identity of the units of the polymer and an order of the units in memory. The computing system may contain program instructions executable to associate the received fluorescent or optical signals with DNA bases.

The reservoir can receive and hold the polymer. The polymer may be a nucleic acid of at least ten thousand bases in length, and the electrodes may be operable to drive the nucleic acid through the channel and pass each of the ten thousand bases, in series, through the exit at the enhancement structure. The device may include a detector to receive fluorescent or optical signals emitted from the nucleic acid at the exit. In some embodiments, a computing system is operably coupled to the detector, the computing system operable to read the signals and write and store the identity of the ten thousand bases of the nucleic acid as one sequence read.

Aspects of the invention provide a method that includes loading a sample that includes a nucleic acid comprising at least ten thousand bases into a reservoir of a device; applying, by electrodes of the device, an electric field to the device to drive the nucleic acid from the reservoir into a nanochannel connected to the reservoir and to a nanochannel exit positioned adjacent a metal-insulator-metal (MIM) enhancement structure; and optically reading via a detector of the device an identify and position within the nucleic acid of each of the ten thousand bases as the nucleic acid emerges from the exit. Preferably, the enhancement structure enhances an electromagnetic field or wave at the exit. In some embodiments, each of the ten thousand bases emits an optical signal in response to stimulus enhanced by the enhancement structure. The optical signal may include fluorescence, interference, or scattering. The method may include writing to memory of a computer system operably coupled to the detector the identity and position of the bases as a sequence read. The method may include comparing, by the computer system, the sequence read to reference data for a human genome and identifying a structural variant in nucleic acid relative to the human genome. In certain embodiments, a first end of the sequence read includes sequence from a first telomere of a first end of a chromosome from a human and a second of the sequence read includes sequence from a second telomere from a second end of the chromosome. Preferably, the nucleic acid is a single DNA molecule and all ten thousand bases are read in one continuous operation.

The nanochannel may have a length between five and ten mm and a cross-section between twenty and ninety nm. The device may include a substructure to direct an ssDNA to a hot spot of the enhancement structure. The device may include a porous silica film positioned at the end of the nanochannel with the enhancement structure atop the porous silica film, and a coating over the porous silica film defining an opening at an edge of the enhancement structure.

Optionally the enhancement structure comprises an elliptical metal disk that includes metal-insulator-metal stack. In some embodiments, the metal is aluminum and the insulator is one of SiO₂ or Al₂O₃.

The method may include providing, via an illumination source, an optical stimulus that is enhanced by the enhancement structure. Materials of the enhancement structure may have dipole resonances at wavelengths of the optical stimulus and at Raman shifted wavelengths of DNA nucleotides.

Other aspects of the invention provide a nanofabricated device for surface enhanced Raman scattering readout of individual nucleotides of a ssDNA strand, the device comprising: a loading zone for loading a solution comprising ssDNA onto the device; a set of electrodes for applying an electric field for electrophoretic control of the ssDNA translocation; a nanopillar array for aiding in loading the ssDNA into nanochannels; one or more nanochannels for extending and linearizing the ssDNA; and an enhancement structure at the exit of each nanochannel for enhancing local electromagnetic fields to excite surface enhanced Raman scattering as nucleotides of an ssDNA strand pass through a hot spot of the enhancement structure. The device may include a substructure to direct the ssDNA to the hot spot of the enhancement structure. The device may include a linear array of nanopillars at the output of the nanochannel and a second set of nanochannels and enhancement structures to provide a second readout of the ssDNA sequence. The nanochannels may be thirty to sixty nm in cross section dimensions and the length is about five to ten mm. the substructure to direct the ssDNA to the hot spot of the enhancement structure may include a porous silica film positioned at the end of the nanochannel; an enhancement structure atop the porous silica film; a coating over the exposed porous silica film with an opening within a small region at the edge of the enhancement structure. The enhancement structure may be an elliptical metal disk. Optionally the enhancement structure is an elliptical metal-insulator-metal stack. Preferably the metal is aluminum and the insulator is one of SiO2 or Al2O3. In some embodiments, the dimensions of the enhancement structure are adjusted to place the dipole resonances of the enhancement structure near the wavelengths of the pump illumination source and the Raman shifted wavelengths of the DNA nucleotides. An opening at the edge of the enhancement structure may be self-aligned to the edge of the enhancement structure by a shadow deposition process. Preferably, maximum linear dimension of the opening at the edge of the enhancement structure is less than ten nm.

In some embodiments, the one or more nanochannels extend through a first chip, wherein the first is mounted on a carrier chip. The loading zone and/or the electrodes may be in the carrier chip. The device may include a detector operable to detect surface enhanced Raman scattering from nucleotides of the ssDNA strand.

Aspects of the invention provide a nanofabricated device for fluorescence readout of dsDNA strands with encoded data bits that are multiple nucleotides in extent, the device comprising: a loading zone for loading the dsDNA in a buffer solution; a set of electrodes for applying a low frequency electric field for electrophoretic control of the dsDNA translocation; a nanopillar array for aiding in loading the dsDNA into nanochannels; a set of nanochannels for stretching the dsDNA into a generally linear conformation; and an enhancement structure at the exit of each channel for providing enhanced local electromagnetic fields to excite fluorescence from the encoded data bits along the dsDNA strands. The device may include a linear array of nanopillars at the output of the nanochannel and a second set of nanochannels and enhancement structures to provide a second readout of the data encoded in the dsDNA. In some embodiments, the nanochannels are about thirty to sixty nm in cross section dimensions and about five to ten mm in length. Preferably, the enhancement structure is elliptical in top down view and is positioned and rotated relative to the nanochannel axis to locate the hot spot of the enhancement structure close to the exit of the nanochannel. A section of the nanochannels may be loaded with silica nanoparticles to slow the dsDNA translocation. The silica nanoparticles may be approximately uniform in size and chosen to fill the nanochannels with gaps between the particles and the nanochannel sidewalls of nm or less. A section of the nanochannels may be filled with a porous silica matrix to slow the DNA translocation. In certain embodiments, the enhancement structure is in-line with the nanochannel which continues both before and after the enhancement structure. The enhancement structure may be offset from the center line of the nanochannel before the enhancement structure. The set of nanochannels may be in a chip mounted onto a carrier chip. The device may include a detector operable to detect the fluorescence from the encoded data bits along the dsDNA strands.

### BRIEF DESCRIPTION OF THE DRAWINGS

FIG. 1 is a top view of structure of a polymer sequencing apparatus.

FIG. 2 is a side view of the apparatus.

FIG. 3 is a top view of the nanochannel and enhancement structure.

FIG. 4 is a side view, showing the enhancement structure.

FIG. 5 shows the use of silica nanoparticles in a nanochannel.

FIG. 6 shows a single row of nanoparticles in a trench deposited by spin coating

FIG. 7 is a top view of a device.

FIG. 8 is a side view of the device.

FIG. 9 is a top view of a first step in a fabrication process.

FIG. 10 is a side view of the step in the process.

FIG. 11 is a top view a of a second step in the fabrication process.

FIG. 12 is a side view of the second step.

FIG. 13 is a top view of a third step of the fabrication process.

FIG. 14 is a side view of the third step.

FIG. 15 is top view of a fourth step.

FIG. 16 is a side view of the fourth step.

FIG. 17 is a top view of the lift-off step of the fabrication process.

FIG. 18 is a side view of the lift-off step.

FIG. 19 is a top view of a shadow evaporation step.

FIG. 20 is a side view of the shadow evaporation step.

FIG. 21 is a top view of a nanochannel with an enhancement structure.

FIG. 22 is a side view of the enhancement structure.

FIG. 23 is a top view of a first step of the in-channel fabrication process.

FIG. 24 is a side view of the first step.

FIG. 25 is a top of view of a second step of the in-channel fabrication process.

FIG. 26 is a side view of the second step.

FIG. 27 shows a third step that involves deposition.

FIG. 28 is a side view of the third step.

FIG. 29 is a top view of a fourth step of the in-channel fabrication process.

FIG. 30 is a side view of the fourth step.

FIG. 31 is top view of a fifth step of the in-channel fabrication process.

FIG. 32 is a side view of the fifth step.

FIG. 33 is a top view of a sixth step of the in-channel fabrication process.

FIG. 34 is a side view of the sixth, planarization step.

FIG. 35 shows a first carrier chip embodiment.

FIG. 36 illustrates a second carrier chip embodiment.

### DETAILED DESCRIPTION

The invention provides methods and devices for determining the sequences of polymers such as biopolymers including nucleic acids. Methods and device are used for determining the identity and order of units of large biopolymers. Device of the invention translocate a molecule such as a nucleic acid through a nanochannel and pass the molecule, base-by-base, by an enhancement structure that enhances an incoming optical stimulus such as an electromagnetic field or wave. The identify of a subunit of the polymer (such as a base of a nucleic acid) as that submit passes by the enhancement structure, has a characteristic effect on the enhanced optical stimulus, such as a characteristic fluorescence or Raman scattering. The characteristic effect of each subunit may be read by a detector to determine the sequence of the molecule. The polymer may be translocated through the channel by electrophoresis (e.g., by applying an electric field), by pressure (e.g., pumping a liquid through), by pulling (e.g., from a distal end of the polymer), enzymatically, or by any other suitable means.

In one embodiment of this invention, a dsDNA strand is directed through a nanochannel, an enhancement structure is placed at the exit of the nanochannel to provide optical readout. A low frequency electric field is used to control the motion of the dsDNA molecule along the nanochannel and past the enhancement structure. This structure is suitable for data storage applications.

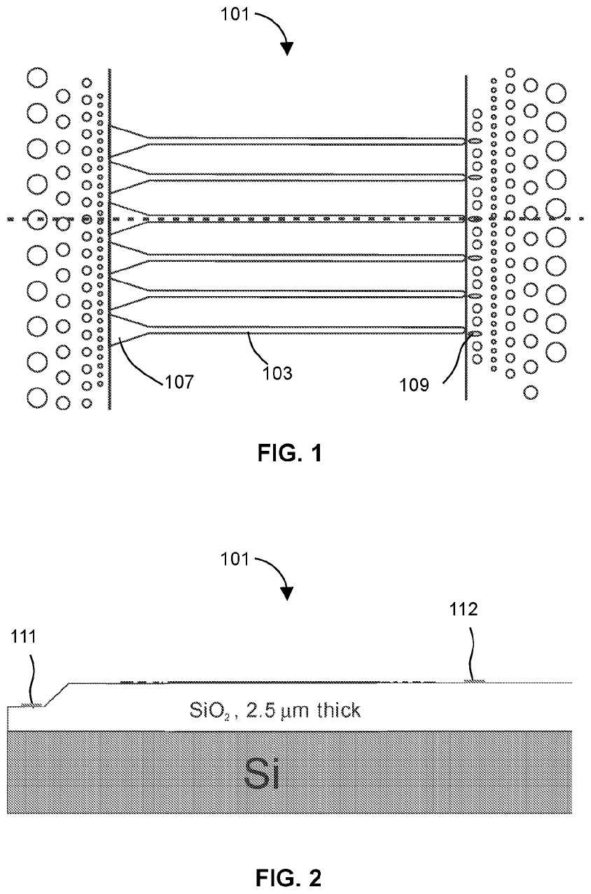

FIG. 1 is a top view of structure of a polymer sequencing apparatus 101. The apparatus includes at least one nanochannel 103. The nanochannel 103 may be, e.g., about 60 nm wide. If a plurality of parallel nanochannels are included, the nanochannels may be on 2 micrometer centers.

FIG. 2 is a side view of the apparatus 101. The structure is fabricated in an approximately 1 to 2.5 mm thick oxide layer atop a silicon wafer. The oxide layer can be a thermal oxide or can be a deposited film; the thick oxide is used to electrically isolate the silicon substrate from the fields applied to transport the dsDNA.

A liquid sample such as a solution that includes nucleic acid (e.g., a dsDNA in buffer solution) may introduced on the left. The device 101 may include contacts 111, 112 (e.g., metal contacts) that may be used to apply a low frequency electric field to move nucleic acid by electrophoresis from left to right and control the transport velocity. The nanochannels are etched into the SiO2 layer with a funnel region 107 at the loading end to enhance dsDNA capture. Typical dimensions for the straight regions of the nanochannels are 50- to 200-nm wide and ˜50- to 200-nm deep to force linearization of the dsDNA. The separation of the nanochannels is 1- to 2-mm to allow optical resolution of DNA in individual nanochannels. The arrays of silica pillars to the left, and optionally to the right, of the nanochannels provide a ‘maze’ that assists in linearizing the dsDNA. An electromagnetic enhancement structure 109 may be placed at the right-hand exit of each nanochannel to provide the sensing of the bits encoded into the dsDNA. A roof (not shown) is bonded to the top of the structure. A suitable cover may be, for example, an approximately 100 to 200-nm thick quartz cover slip. Optionally an additional nanochannel section and sensor could be placed to the right to provide a redundant read-out to improve the accuracy.

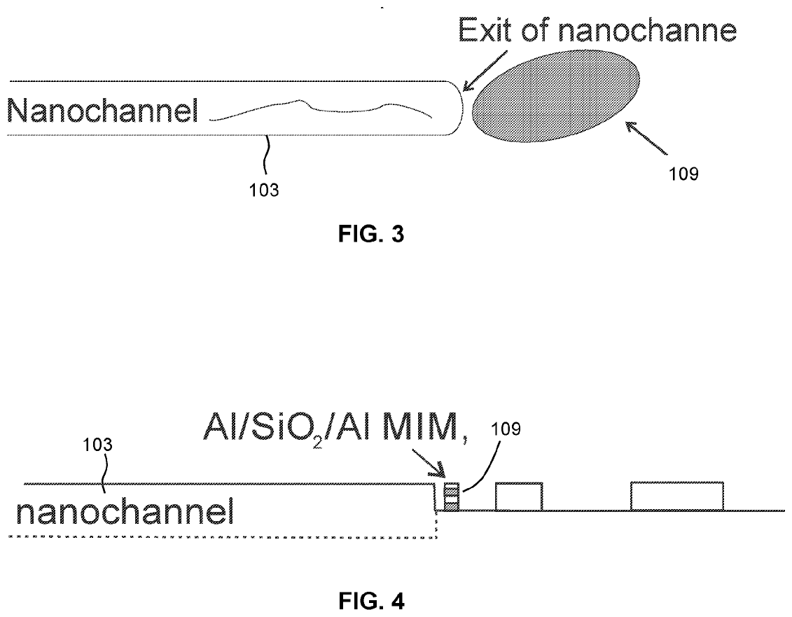

FIG. 3 is a top view of the nanochannel 103 and enhancement structure 109. The enhancement structure 109 is shown as an elliptical cross section that is rotated ˜15° from the axis of the nanochannel.

FIG. 4 is a side view, showing an exemplary enhancement structure 109 as a metal-insulator-metal (Al/SiO₂/Al) structure. See US patent publication US 2022/0244186 A1, incorporated by reference. Gold (Au) provides excellent enhancement. Aluminum (Al) may be preferred for its compatibility with semiconductor manufacturing. The optimal dimensions, sub-structure and positioning of the enhancement structure will depend on the details of the nanochannel dimensions and bit sizes along the dsDNA and on the excitation and fluorescence wavelengths. For an elliptical structure, the maximum enhancement is obtained when the incident laser excitation is polarized along the long axis of the ellipse.

Optionally, a segment of the nanochannel can be packed with a porous media to further restrict the transport velocity of a polymer such as dsDNA.

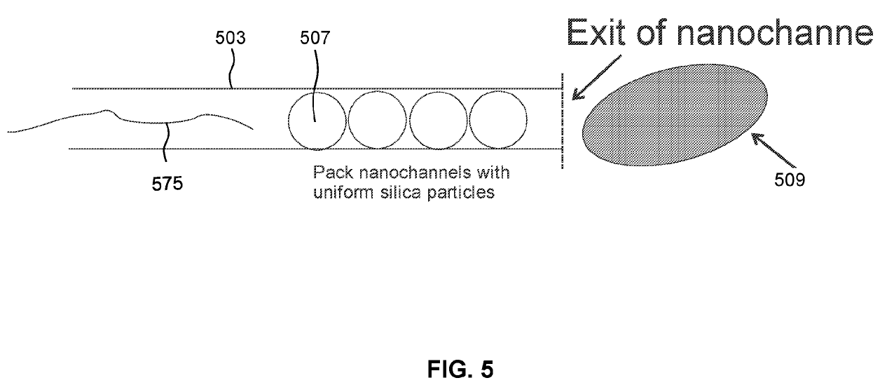

FIG. 5 shows the use of silica nanoparticles507 in a nanochannel 503 that terminates at an enhancement structure 509. A polymer 575 is in the nanochannel 503 (e.g., a strand of DNA). The particles 507 may be deposited in a spin coating step as has been demonstrated. Uniformly sized silica nanoparticles are commercially available. The size is preferably chosen such that the particles fit within the nanochannels with only small gaps less than or approximately 5 nm on each side. This provides a tortuous pathway that will slow the dsDNA translocation.

Alternatively, a porous film could be deposited in the nanochannel. See Uchida, 2000, Chemical vapor deposition based preparation on porous silica films, Jpn J Appl Phys 39: L1155-L1157, incorporated by reference.

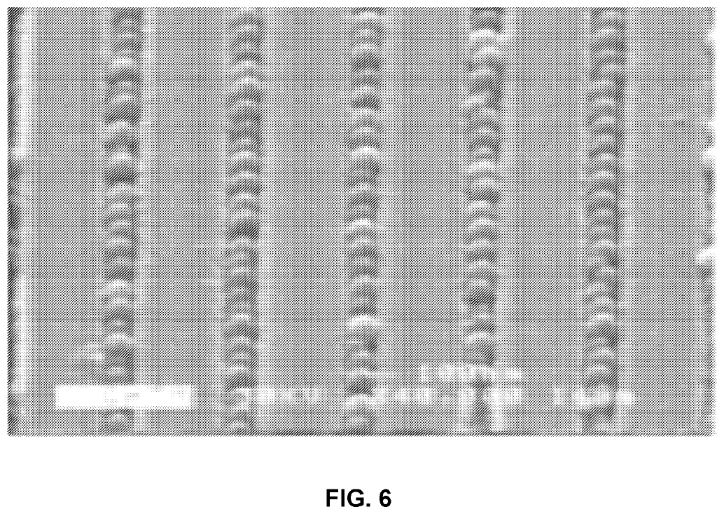

FIG. 6 shows a single row of nanoparticles in a trench deposited by spin coating. See Xia, 2004, Directed self-assembly of silica nanoparticles into nanometer-scale patterned surfaces using spin-coating, Adv Mat 16:1427, incorporated by reference.

Significant particle size dispersion is evident, more uniform particles are available. Assuming a 60 nm rectangular channel and 50 nm silica particles, this will result in a series of short (10 s of nm) approximately 10 nm tunnels that will force the ssDNA to take a convoluted path past the nanoparticles and substantially slow the ssDNA translocation.

In some embodiments, a ssDNA strand is directed through a nanochannel, an enhancement structure is placed at the exit of the nanochannel to provide optical readout. A low frequency electric field is used to control the motion of the ssDNA molecule along the nanochannel and past the enhancement structure. This structure is suitable for DNA sequencing applications. In such embodiments, a ssDNA strand is used to provide access to sequencing data. The placement of the enhancement structure is important because much higher spatial resolution is required as a single nucleotide is only ˜0.34 nm long. In the fourth embodiment, the nanochannel is packed with either silica nanoparticles or a porous film.

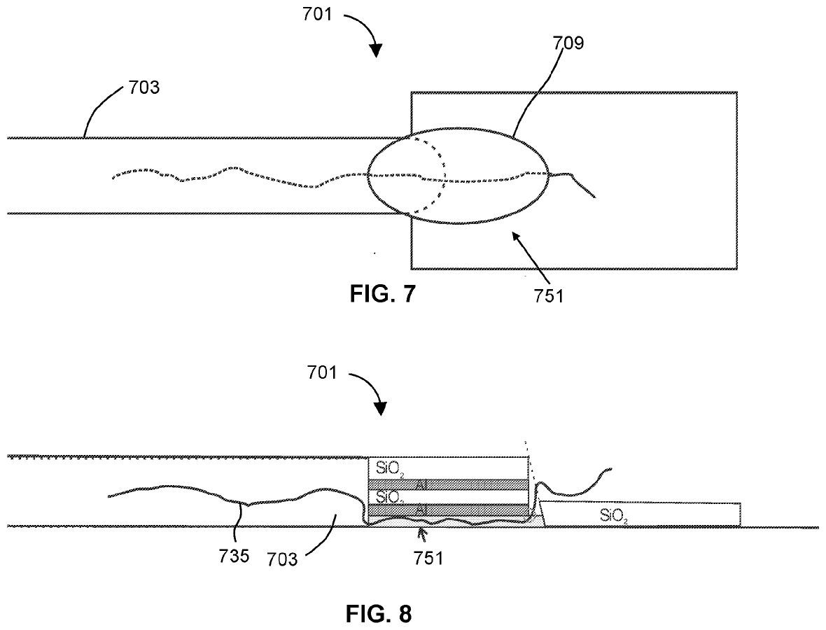

FIG. 7 is a top view of a device 701 with a nanochannel 703 and a porous silica film 751 underneath an enhancement structure 709.

FIG. 8 is a side view of the device 701, which provides a fifth embodiment of the invention. As shown, a section near the end of the nanochannel 703 is filled with a porous silica film 751 and the enhancement structure 709 is placed atop this porous silica, forcing the nucleic acid 735 (e.g., ssDNA) to pass by the hot spot at the edge of the enhancement structure. The figure depicts an Al/SiO2/Al MIM enhancement structure placed at the end of the nanochannel. Importantly, this is not a critical alignment, the MIM can move horizontally by 10- to 20-nm without affecting the sequencing performance. The MIM is supported atop a porous silica film that slows the ssDNA translocation. The dimensions (ellipse size, aspect ratio and film thicknesses) are chosen to adjust the dipole resonances of the MIM to the vicinity of the illumination and scattering wavelengths. A SiO2 deposition on top of the MIM brings the height of the structure close to the top of the nanochannel walls to allow the coverslip sealing, not shown. The ssDNA emerges from the porous silica film just at the end of the MIM (hot spot indicated with a red circle. A block is self-aligned to the MIM ensuring that the ssDNA emerges vertically in the vicinity of the hot spot. A fabrication sequence is outlined below.

In operation, the ssDNA is introduced on the left, is driven by electrophoresis past the nanopillar array and enters the nanochannel. At the right end of the nanochannel, the ssDNA is forced to enter the porous silica film since the rest of the nanochannel is blocked by the MIM structure.

The DNA traverses the porous silica film, which slows the translocation and exits through a narrow, self-aligned region just in the vicinity of the hot spot of the elliptical structure. Because the hot spot is highly localized in both the vertical and horizontal directions of the figure, only one or two nucleotides are in the hot spot at any time, allowing unambiguous readout of the nucleotides with surface-enhanced Raman scattering. This allows identification of epigenetic variations, which is not possible with any other available technologies.

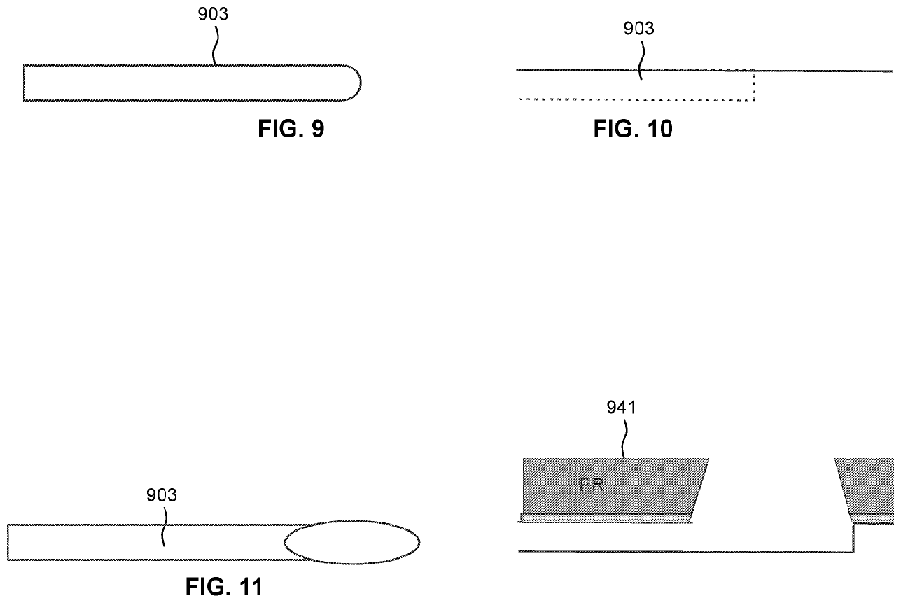

FIG. 9 through FIG. 20 show steps of a fabrication process with self-aligned features.

FIG. 9 is a top view of a first step in a fabrication process for making a structure that includes a nanochannel 903. The first step of the process is to fabricate and etch the nanochannels 903 into the silicon dioxide layer atop the silicon wafer. There are multiple approaches to this process, using both positive and negative tone resists and all are incorporated here by reference.

FIG. 10 is a side view of the step in the process. The process shown is based on a lift-off process where materials are deposited through an opening in a photoresist film and the film is then removed. There are analogous processes starting from blanket films that are often more appropriate for semiconductor manufacturing facilities. These are not show explicitly but are incorporated by reference.

FIG. 11 is a top view a of a second step in the fabrication process. The purpose of the second step is to deposit blanket films of bottom anti-reflection coating and of photoresist (PR) 941 and to open a hole over the location for the enhancement structure. The exposure/development process for the photoresist 941 is designed to produce a slight undercut 945, shown as a ˜15° sidewall slope in the figure.

FIG. 12 is a side view of the second step, showing the undercut 945 in the photoresist 941.

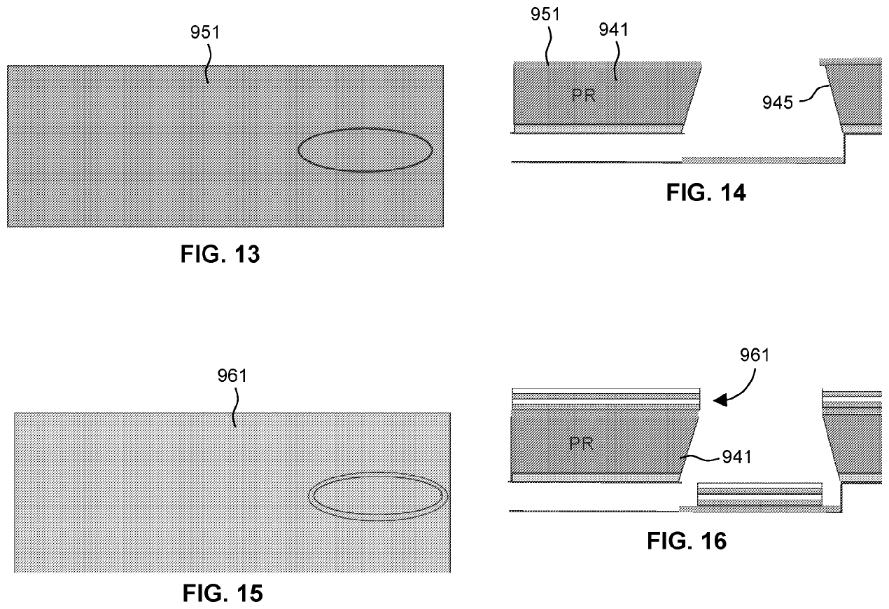

FIG. 13 is a top view of a third step of the fabrication process, in which a porous silica film is deposited, e.g., by chemical vapor deposition.

FIG. 14 is a side view of the third step in which the porous silica film 951 is deposited by chemical vapor deposition. The depicted fabrication process allows the deposited film to extend beyond the opening at the top of the photoresist 941 and to extend to the limits of the larger bottom opening.

FIG. 15 is top view of a fourth step of the fabrication process, showing the deposition of a film stack 961.

FIG. 16 is a side view of the fourth step in which the remaining films of the enhancement structure, shown as an Al—SiO2—Al—SiO2 film stack 961 are deposited by directional processes such as sputtering or e-beam evaporation. Dimensions of the film stack 961 are restricted by the dimensions of the top opening in the photoresist 941.

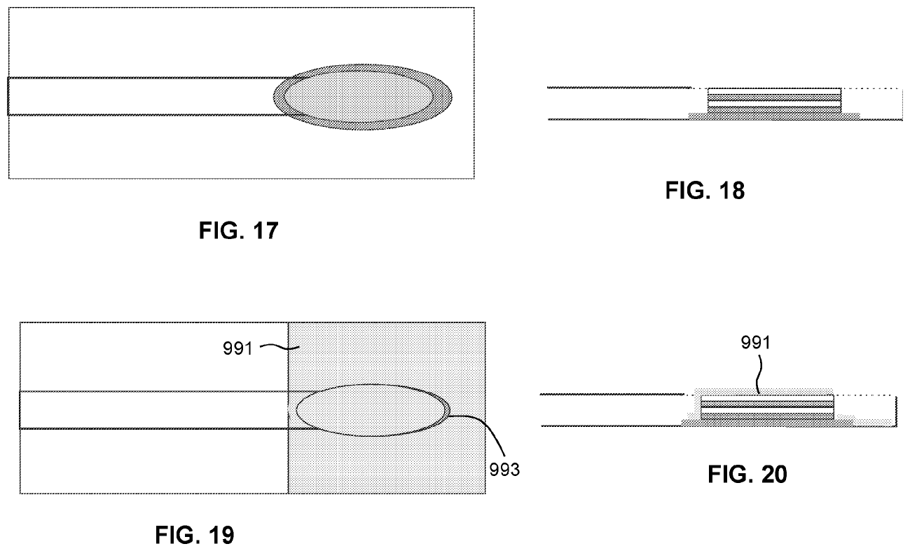

FIG. 17 through FIG. 20 show the final steps in the fabrication process. After the films are deposited to form the film stack 961, the photoresist and bottom ARC are removed, in a lift-off process.

FIG. 17 is a top view of the lift-off step of the fabrication process.

FIG. 18 is a side view of the lift-off step. After the lift-off step, a silicon dioxide film will be deposited.

FIG. 19 is a top view of a shadow evaporation step of the fabrication process, showing deposition of a silicon dioxide film 191.

FIG. 20 is a side view of the shadow evaporation step. The silicon dioxide film 991 is deposited with a directional process such as e-beam evaporation. This is known as a shadow evaporation. Preferred embodiments use a 10° angle of incidence of the beam. The deposited material is shown as a thin silicon dioxide film 991. The result is to leave a small crescent 993 of the porous material open just at the hot spot of the enhancement structure 909.

Other geometries, arrangements, and embodiments are within the scope of the disclosure. For example, some embodiments use one or more enhancement structures that are within, or in line with, or sitting along the length of, one of more nanochannels.

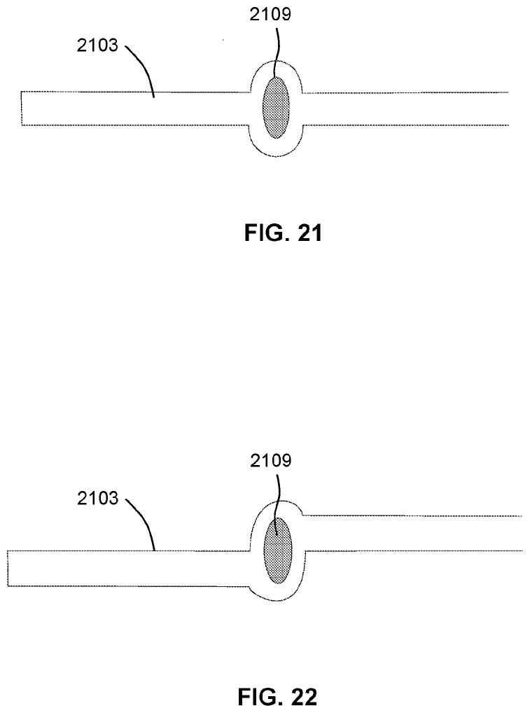

FIG. 21 is a top view of a nanochannel with an enhancement structure 2109 placed in line with the nanochannel 2103.

FIG. 22 is a side view of the enhancement structure 2109 is placed in line with the nanochannel 2103. The enhancement structure 2109 is shown approximately centered in the nanochannel 2013. Since the fabrication of the nanochannel occurs in a separate lithographic step from the placement of the enhancement structure, shown as an ellipse in this figure, the placement accuracy is limited by lithographic considerations to plus/minus 10 nm, which can be significant with a channel width of ˜50 nm. Depending on the specific application, the elliptical enhancement structure can be oriented with the long axis either perpendicular to the nanochannel flow direction as shown in FIG. 21, or parallel to the flow direction (not shown).

In the sequencing application it is necessary to provide a localization structure such that ssDNA is forced to the hot spot of the MIM structure.

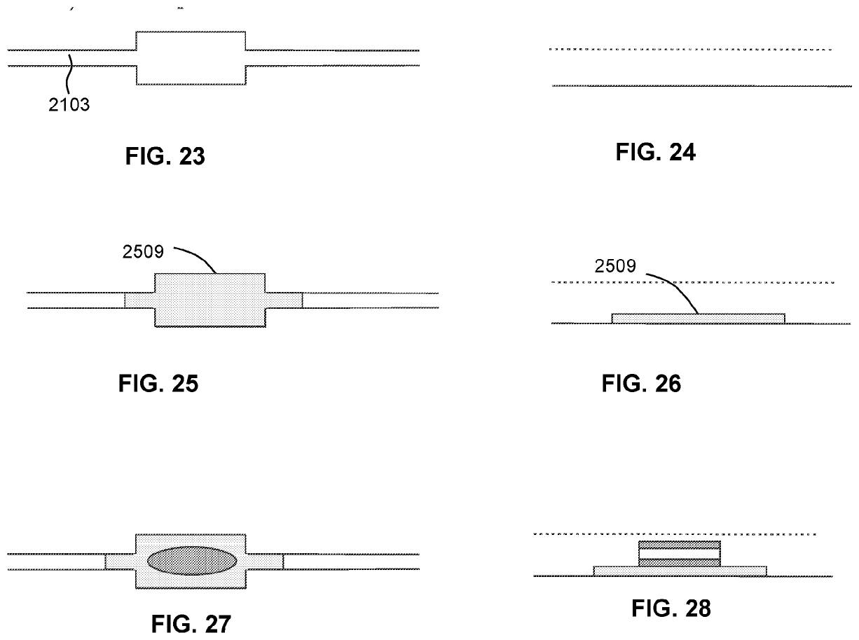

FIG. 23 through FIG. 34 show steps an in-channel fabrication process to form a device with an enhancement structure 2109 in a nanochannel 2103.

FIG. 23 is a top view of a first step of the in-channel fabrication process. In the in-channel fabrication process, a lithography and etch sequence is performed to etch the nanochannels 2105 into the ˜0.5 to 1 micrometer SiO2 layer over the Si wafer (not shown). Provision is made to include an enhancement structure in-line with the nanochannel in subsequent process steps.

FIG. 24 is a side view of the first step.

FIG. 25 is a top of view of a second step of the in-channel fabrication process, the second step being a second lithography and deposition step, in which a porous layer 2509 is deposited into the region for the enhancement structure extending some distance into the nanochannels to both sides of the enhancement structure region.

FIG. 26 is a side view of the second step, showing the porous layer 2509 created by the deposition. The purpose of this deposition is to provide a medium that slows the ssDNA translocation. This could be either a porous layer or a mesoporous layer, characterized by a periodic array of uniform nanopores.

FIG. 27 shows a third step that involves deposition of porous SiO2 around a metal-insulator-metal (MIM) area using a photoresist mask. The deposition area can be larger than that shown before with some material deposited on the walls between the nanochannels; excess material can be removed in a subsequent planarization step.

FIG. 28 is a side view of the third step. The enhancement structure is deposited with an additional lithographic masking. As drawn, an elliptical metal-insulator-metal-insulator (MIM) structure is shown along with an additional insulator layer to match depth of the nanochannels; the details of the enhancement structure are to be adjusted for optimal surface-enhanced Raman scattering (SERS) sensitivity. In addition, the thickness of the final layer can result in the top surface of the enhancement structure being above the top of the nanochannels; this excess material can be removed in a subsequent planarization step.

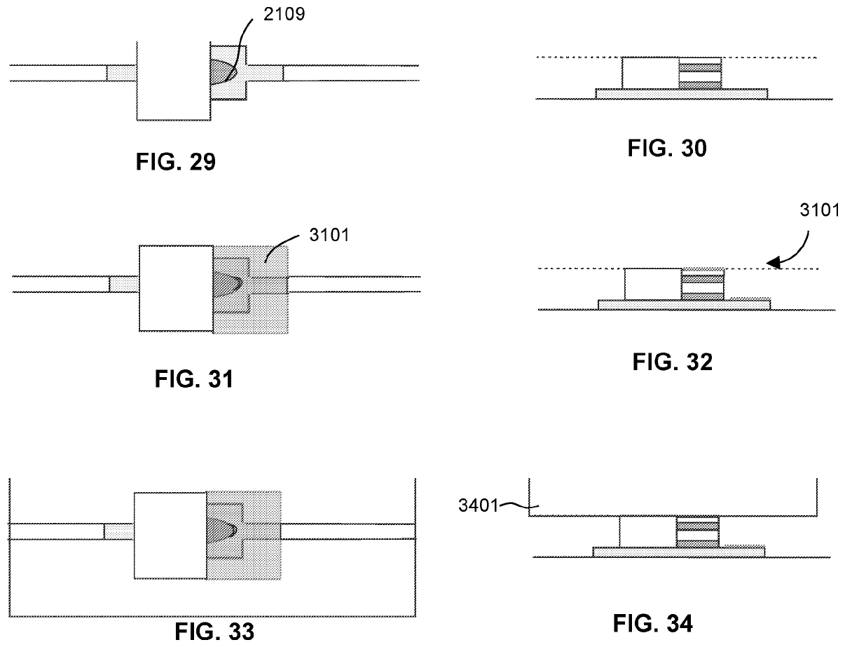

FIG. 29 is a top view of a fourth step of the in-channel fabrication process. The fourth step provides for sealing a front of a MIM enhancement structure 2109 to force a polymer such as nucleic acid (e.g., DNA) into p-SiO2 additional photoresist mask. FIG. 30 is a side view of the fourth step. With an additional masking step, SiO2 or similar material is deposited to seal the nanochannel/enhancement structure interface and force the ssDNA to translocate through the porous material under the enhancement structure. Again, excess material can be removed in a subsequent planarization step. The effects of this material on the resonance properties of the enhancement structure have to be taken into account in the design of the enhancement structure.

FIG. 31 is top view of a fifth step of the in-channel fabrication process. The fifth step provides a shadow mask layer to self-align gap with MIM hot spot, additional photoresist mask. Specifically, a dielectric layer 3101, for example Si3N4 or Al2O3 is deposited with a shadow deposition to seal the top of the exposed porous layer.

FIG. 32 is a side view of the fifth step. The shadow deposition results in an unsealed, self-aligned region directly adjacent, within a few nm, to the hot spot of the enhancement structure.

FIG. 33 is a top view of a sixth step of the in-channel fabrication process. The sixth step is a planarization step that is performed to provide a flat top surface.

FIG. 34 is a side view of the sixth, planarization step. As shown, a cover slip 3401 is anodically bonded to provide a roof for the nanochannels.

It may be advantageous to provide a carrier for the chip to allow for sample introduction and the application of a de or dynamic electric field to assist in controlling the ssDNA translocation past the hot spot of the enhancement structure.

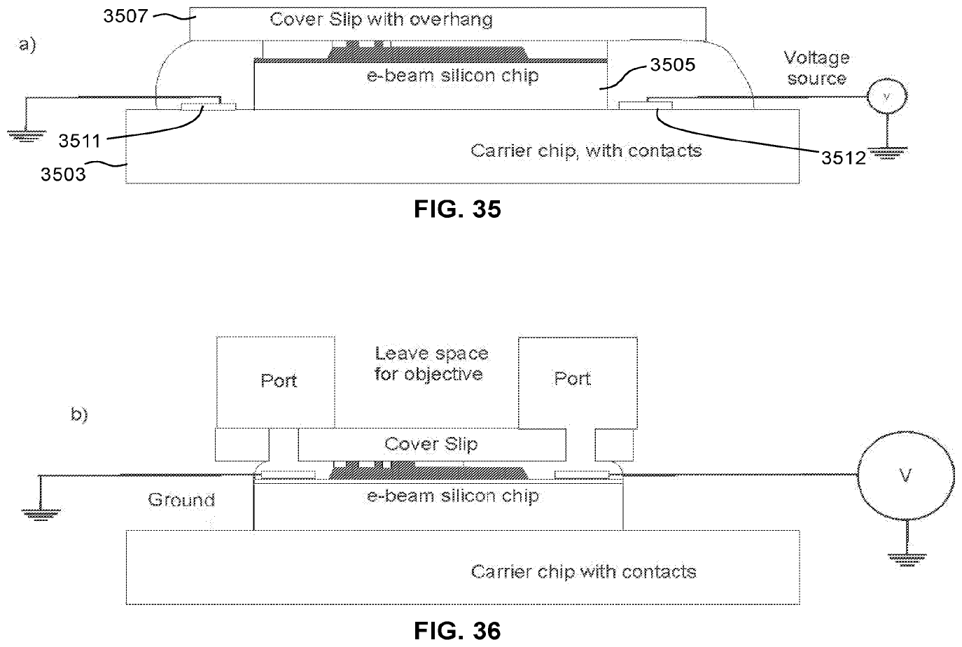

FIG. 35 shows a first carrier chip embodiment, in which the silicon chip 3505 is mounted on a carrier 3503. The carrier has contact pads 3511, 3512 that allow for the application of an electric field that can be varied to affect translocation of a nucleic acid such as ssDNA. The cover slip 3507 has an overhang beyond the silicon chip 3505 to allow for the introduction of a liquid sample, such as a solution that includes a nucleic acid, to the nanochannels.

FIG. 36 illustrates a second carrier chip embodiment, in which ports are shown attached to through holes drilled into the cover slip. In this example the contact pads have been moved to the silicon chip. The disclosure include variations of the depicted examples.

Devices and structures of the disclosure are useful for capturing, holding, elongating (e.g. straightening), translocating, and analyzing polymers such as nucleic acids. For example, a solution that includes a nucleic acid may be transferred into a receiving reservoir, well, or end of a channel and the nucleic acid may be driving into the nanochannel, causing the nucleic acid molecule to elongate and extend through the nanochannel, whether essentially straight or tortuous. The molecule may be driven through the channel and drive to emerge at or pass near an MIM enhancement structure. Chemical identity of the molecule may be read as it emerges at or passes near the enhancement structure. For example, the molecule can be probed with light and scattering, reflection, distortion, refraction, interference, shifting, or some other such effect may be detected from the light that has interacted with the molecule. E.g., a light detector (complex-metal oxide semiconductor sensor, a photodiode, a photovoltaic device, or other such sensor) may be used to detect or measure an effect on the light of the molecule. In fact, it may be that each base of a nucleic acid will have a characteristic effect light, e.g., characteristic Raman scattering pattern, for example. A detector can read the characteristic light and a connected computing device can record the identity of each nucleic acid base as the molecule emerges form or passes near the enhancement structure. By such means, devices and methods of the invention are useful to read the order sequence of a bases of a polymer such as nucleic acid molecule. Accordingly, methods and device of the disclosure are useful for sequencing bases on a DNA chain. For sequencing, individual bases are to be read in sequence. For data storage applications, bits can be larger consisting of multiple bases and additional moieties including dyes for labeling.

Device and methods of the invention are useful for a variety of specific chemistries, circumstances, and applications.

For example, device and methods of the invention are useful for the analysis of genomic structural variants (SVs). The association of sequence variations in a variety of human diseases is well-understood. However, short read detection of sequence variation is limited to single-nucleotide variants or small insertions and deletions and is not suited for the structural variants (e.g. duplications, inversions, or translocations that affect >50 bp) that comprise the majority of variation in the human genome. Device and methods of the invention provide the necessary context to anchor and sequence resolve most SVs, allowing better understanding of disease associations.

Device and methods of the invention are useful for haplotype-resolved sequencing. The principal advantage of haplotype-resolved sequencing with a long read approach is that all polymorphisms are assigned to a specific chromosome (i.e., maternal vs. paternal), and links are established between mutations (or variants) in distant regulatory elements and cis-linked genes on the same chromosome, yielding valuable elucidation of disease states. The few approaches attempted to date to provide haplotype information by short read sequencing method add significant complexity and cost associated from upstream processing. The HapMap and other projects are developing a haplotype map, but new approaches are required to address the cis and trans relationships in variants that occur in rare genotypes (e.g., novel somatic mutations) or in altered genomes (e.g., cancer).

Device and methods of the invention are useful address existing challenges associated with the de novo whole genome assembly with current short read technologies, where the assembly of short reads requires tremendous computational resource, and the quality of the resulting genome assembly is low relative to re-sequencing projects or projects that incorporate long read technologies. The need to align to a reference genome for assembly severely limits identification of complex SVs. Device and methods of the invention are useful to fill gaps and provide scaffolding for short reads and resolution of repetitive regions.

Epigenetics Bisulfite short read sequencing has been the gold standard for mapping methylated cytosines but suffers from several limitations, including the inability to assay repetitive genomic regions. Device and methods of the invention overcome those limitations, where currently available technologies come up short due to insufficient resolution. Direct optical measurement of Raman signatures of single-base epigenetic modifications, with long reads, provides the necessary information for characterization of epigenetic chemical variations.

Device and methods of the invention are useful for long-read single-molecule sequencing. Prior attempts to achieve the long read goal included using TEM imaging (Halcyon Molecular, ZS Genetics), optical imaging of long labeled DNA in nanochannels (Bionano Genomics), and several approaches that combine nanopores, DNA processing enzymes and various readout systems (Base4, Genia). Other attempts include linked read (e.g., 10× Genomics) and synthetic long read technologies. Long read technologies include those by Pacific Biosciences (PacBio) and Oxford Nanopore Technologies (ONT). Device and methods of the invention are useful address primary challenges associated with nanopore sequencing including resolution and throughput. Existing long read technologies move the DNA molecule too fast to resolve individual nucleotides. Electrical readout is incompatible with the massive parallelization that would be required for good throughput from those platforms. Device and methods of the invention are useful to slow the DNA translocation to get single-base resolution and use optical (e.g., Raman) detection for very high throughput.

Device and methods of the invention are useful for data storage applications The needs for data storage are increasing exponentially, figuring out where and how to store it efficiently and inexpensively becomes a larger problem every day. The prevailing long-term method, which dates from the 1950s, writes data to pizza-sized reels of magnetic tape. By comparison, DNA storage is potentially less expensive, more energy-efficient and longer lasting. Studies show that DNA properly encapsulated with a salt remains stable for decades at room temperature and should last much longer in the controlled environs of a data center. DNA doesn't require maintenance, and files stored in DNA are easily copied for negligible cost. Even better, DNA can archive a staggering amount of information in an almost inconceivable small volume. Consider this: humanity will generate an estimated 3.3×1022 bytes of data by 2025. DNA storage can squeeze all that information into a ping-pong ball, with room to spare. The 74 million million bytes of information in the Library of Congress could be crammed into a DNA archive the size of a poppy seed—6,000 times over. Split the seed in half, and you could store all of Facebook's data. Device and methods of the invention are useful for rapidly reading data that has been stored in DNA.

The invention provides methods and device for determining the identity and order of units of large biopolymers. Device of the invention use electrical fields to translocate the molecule through the channel and pass the molecule, base-by-base, by an enhancement structure that enhances an incoming optical stimulus such as an electromagnetic field or wave. The presence of a DNA bases at the exit, adjacent the enhancement structure, has a characteristic effect in response to the enhanced optical stimulus, such as a characteristic fluorescence or Raman scattering. The characteristic effect of each base is read by the detector to determine the sequence of the molecule.

1. A device for reading units of a polymer, the device comprising:
a loading reservoir for receiving a sample that includes a polymer;
a channel extending from the reservoir;
electrodes operable to generate a field to drive the polymer from the reservoir and through the channel, wherein the polymer assumes an elongated conformation in the channel;
an enhancement structure at an exit of the channel to enhance electromagnetic fields at the exit and excite fluorescence from the polymer.

2. The device of claim 1, further comprising a detector to receive fluorescent or optical signals emitted from the polymer at the exit.

3. The device of claim 1, further comprising a linear array of nanopillars at the exit of the channel and a second set of nanochannels and enhancement structures to provide a second readout of the data encoded in the polymer.

4. The device of claim 1, wherein the channel is one of a plurality of nanochannels.

5. The device of claim 4, further comprising the polymer in the channel, wherein the polymer is a DNA molecule.

6. The device of claim 5, further comprising a funnel structure and/or a nanopillar array at the reservoir for aiding in loading the DNA into the nanochannel.

7. The device of claim 4, wherein the nanochannels have a cross-sectional dimension between about 30 and 60 nm and a length between about 5 and 10 mm.

8-16. (canceled)

16. A method comprising:
loading a sample that includes a nucleic acid comprising at least 10,000 bases into a reservoir of a device;
applying, by electrodes of the device, an electric field to the device to drive the nucleic acid from the reservoir into a nanochannel connected to the reservoir and to a nanochannel exit positioned adjacent a metal-insulator-metal (MIM) enhancement structure; and
optically reading via a detector of the device an identify and position within the nucleic acid of each of the 10,000 bases as the nucleic acid emerges from the exit.

17. The method of claim 16, wherein the enhancement structure enhances an electromagnetic field or wave at the exit.

18. The method of claim 16, wherein each of the 10,000 basis emits an optical signal in response to stimulus enhanced by the enhancement structure.

19. The method of claim 18, wherein optical signal comprise fluorescence, interference, or scattering.

20. The method of claim 16, further comprising writing to memory of a computer system operably coupled to the detector the identity and position of the bases as a sequence read.

21. The method of claim 20, further comprising comparing, by the computer system, the sequence read to reference data for a human genome and identifying a structural variant in nucleic acid relative to the human genome.

22-30. (canceled)

31. A nanofabricated device for surface enhanced Raman scattering readout of individual nucleotides of a ssDNA strand, the device comprising:
a loading zone for loading a solution comprising ssDNA onto the device;
a set of electrodes for applying an electric field for electrophoretic control of the ssDNA translocation;
a nanopillar array for aiding in loading the ssDNA into nanochannels;
one or more nanochannels for extending and linearizing the ssDNA; and
an enhancement structure at the exit of each nanochannel for enhancing local electromagnetic fields to excite surface enhanced Raman scattering as nucleotides of an ssDNA strand pass through a hot spot of the enhancement structure.

32. The device of claim 31, further comprising a substructure to direct the ssDNA to the hot spot of the enhancement structure.

33. The device of claim 31, further comprising a linear array of nanopillars at the output of the nanochannel and a second set of nanochannels and enhancement structures to provide a second readout of the ssDNA sequence.

34. The device of claim 33, wherein the nanochannels are about 30 to 60 nm in cross section dimensions and have length of about 5 to 10 mm.

35. The device of claim 31, wherein the substructure to direct the ssDNA to the hot spot of the enhancement structure is composed of:
a porous silica film positioned at the end of the nanochannel; an enhancement structure atop the porous silica film;
a coating over the exposed porous silica film with an opening within a small region at the edge of the enhancement structure.

36. The device of claim 31, wherein the enhancement structure is an elliptical metal disk.

37. The device of claim 31, wherein the enhancement structure is an elliptical metal-insulator-metal stack.

38-58. (canceled)
